(19) United States
(12) Patent Application Publication
(10) Pub. No.: US 2025/0000752 A1
(43) Pub. Date: Jan. 2, 2025

(54) Rinse-Off Personal Care Composition

(71) Applicants: Conopco, Inc., d/b/a UNILEVER, Englewood Cliffs, NJ (US); UNILEVER IP HOLDINGS B.V., AL Rotterdam (NL); UNILEVER GLOBAL IP LIMITED, Merseyside (GB)

(72) Inventors: Xiaoyun Pan, Shanghai (CN); Xuezhi TANG, Shanghai (CN); Jinfang WANG, Shanghai (CN)

(21) Appl. No.: 18/709,208

(22) PCT Filed: Oct. 21, 2022
(86) PCT No.: PCT/EP2022/079350

§ 371 Date: May 10, 2024

(30) Foreign Application Priority Data
Nov. 12, 2021 (WO) PCT/CN2021/130217
Dec. 24, 2021 (EP) 21217667.1

(51) Int. Cl. A61K 8/02; A61Q 5/02; A61Q 19/10

(52) U.S. Cl. CPC A61K 8/0266; A61Q 5/02; A61Q 19/10; A61K 2800/41; A61K 2800/651

(57) ABSTRACT

Disclosed is a rinse-off personal care composition comprising: (a) a microcapsule comprising an intercalated layered particle having an interlayer distance of 1 to 10 nm and an active inside of the particle; and (b) an anionic surfactant, wherein the active is selected from natural essential oil, pro-fragrance, fragrance, mosquito repellence active, anti-dandruff active or a mixture thereof.

### FIELD OF THE INVENTION

The present invention relates to a rinse-off personal care composition. In particular, the present invention relates to a rinse-off personal care composition for enhanced foaming efficacy.

### BACKGROUND OF THE INVENTION

Rinse-off personal care products are usually used for cleansing our skin, scalp and hair, for example, skin cleansing products and hair cleansing products. Some consumers prefer more foaming because more foaming suggests that the product is effective in cleansing. In addition to cleansing, some consumers also desire various benefits from their skin and hair cleansing products. Therefore, benefit actives may be added to the formulation. However, some benefit actives may negatively impact foaming such as the amount or the quality of foaming. To improve the performance of benefit actives, encapsulation of those actives in microcapsules has been explored.

Encapsulation refers to the process of encapsulating a substance in a capsule material to form nano-, micro-, or millimeter-sized microcapsules. Traditional microcapsules may have core-shell structure in which the active (the core) is surrounded by the capsule material (the shell). When applied on the skin, scalp or hair, microcapsules may be deposited and then broken by action of pressure and/or friction. Active is then released to bring various benefits, for example, repellents for repelling mosquitoes, anti-dandruff actives for reducing dandruff, and perfume for providing superior sensory.

Surfactants are commonly used in rinse-off personal care products to cause foaming and help clean during use of the product, especially anionic surfactants. However, certain anionic surfactants may form complex with Ca²⁺ from the tap water which also leads to low foaming.

Therefore, there is still room to improve rinse-off personal care products for their foaming efficacy.

The present inventors have developed a rinse-off personal care composition comprising a microcapsule which comprises a particle having an interlayer distance of 1 to 10 nm, an active inside of the particle, and an anionic surfactant. The present inventors have surprisingly found that the rinse-off personal care composition provided enhanced foaming benefit.

### SUMMARY OF THE INVENTION

In a first aspect, the present invention provides a rinse-off personal care composition comprising:
(a) a microcapsule comprising an intercalated layered particle having an interlayer distance of 1 to 10 nm and an active inside of the particle; and
(b) an anionic surfactant, wherein the active is selected from natural essential oil, pro-fragrance, fragrance, mosquito repellence active, anti-dandruff active or a mixture thereof.

In a second aspect, the present invention provides a method of foam enhancement for cleansing skin of an individual comprising the step of applying the composition of the present invention to the skin, scalp or hair of an individual.

In a third aspect, the present invention provides use of the composition of the present invention for providing foam enhancement for cleansing skin, scalp or hair of an individual.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

### Terminology

### Interlayer Distance

Interlayer distance, as used herein, means the distance between the adjacent clay layers, and can be measured by X-ray diffraction (XRD). A suitable method of measuring the interlayer distance is to use Rigaku Miniflex II (Tokyo, Japan) with Cu Kα radiation at 30 kV and 15 mA. The samples are observed in the 20 range of 5-80° at a speed of 2.5°/min (see Colloids and Surfaces A: Physicochem. Eng. Aspects 507, 2016, page 143).

### Sphericity

Sphericity is the measure of how closely the shape of an object approaches that of a mathematically perfect sphere. The sphericity of a particle is the ratio of the surface area of a sphere (with the same volume as the given particle) to the surface area of the particle.

### Log P

Log P, as used herein, means the partition coefficient of a molecule between n-octanol and water. Log P indicates the lipophilicity of a molecule. The higher log P is, the more lipophilic the molecule is.

### Microcapsule

The composition of the present invention comprises a microcapsule. A microcapsule is a small capsule used to contain an active. Microcapsule herein comprises an intercalated layer particle wherein the particle further comprises an active. The active is present in between the interlayers of the particle.

Preferably the microcapsule has platy shape which means it is flat and plate-like. Preferably the microcapsule is not core-shell structure.

A microcapsule may comprise one or more intercalated particles. The microcapsule may be prepared in any suitable process. Commonly used microcapsule preparation methods include the complex coacervation method, the ionic gel method, the microemulsion polymerization method, the layer-by-layer assembly method, the air-suspension method, and the absorption encapsulation method. It is preferable that the process for preparing the microcapsule comprises the step of mixing of the particle with the active at 0 to 40° C. Preferably, the step of mixing is conducted under agitation. The microcapsule preferably comprises one single particle which may be achieved by sonication. The microcapsule has a size preferably from 100 nm to 50 μm, and more preferably from 1 to 20 μm.

Typically, the composition comprises the microcapsules at levels of from 0.001% to 10%, more preferably from 0.005% to 8%, even more preferably from 0.01% to 5% and most preferably from 0.1% to 2% by weight of the total composition.

Preferably, the particle is intercalated layered particle. The preferred shape of the particle is platy. Preferably, the active is evenly distributed in between the interlayers of the particle. For the particles, both synthetic and naturally occurring layer compounds can be used, for example, phyllosilicates or clay minerals or clay mineral containing bentonite, kaolinite, halloysite, sericite, montmorillonite, smectite, hectorite, saponite, talc, vermiculite, allevardite, amesite, beidellite, fluorhectorite, fluorvermiculite, mica, illite, muscovite, nontronite, palygorskite, sepiolite, stevensite, and synthetic types of talc and the alkali silicates maghemite, magadiite, kenyaite, makatite, silinaite, grumantite, revdite and their hydrated forms and the corresponding crystalline silicas or other inorganic layered compounds such as hydrotalcite, double hydroxides and heteropoly acids, preferably phyllosilicates and double hydroxides.

Preferably the particle is intercalated layered clay. It is more preferably that the particle comprise bentonite, kaolinite, halloysite, sericite, montmorillonite, smectite, hectorite, saponite, vermiculite, or talc. The particle is most preferably intercalated bentonite. Such particle can be commercially available from Zhejiang Fenghong new material Co. Ltd.

The particle size may be measured by laser diffraction particle size analyzer, for example, using a system (such as a Mastersizer™ 2000 available from Malvern Instruments Ltd) meeting the requirements set out in ISO 13320. Preferably the particle has a size from 10 to 20 μm. Preferably, the particle has a non-spherical shape. The particle has a sphericity of preferably from 0.01 to 0.74, more preferably from 0.01 to 0.5, and most preferably from 0.01 to 0.15.

The interlayer distance of the particle may be measured by XRD. Preferably, the interlayer distance of the particle is 1 to 10 nm, and more preferably 2 to 8 nm, and most preferably 1 to 4 nm.

The particle is preferably intercalated by fatty ammonium which has the general formula R¹R²R³R⁴N⁺ wherein the R groups are long or short hydrocarbon chains, typically alkyl, hydroxyalkyl or ethoxylated alkyl groups. For example, the fatty ammonium in which R¹ is a C₁₀-C₂₂ alkyl group, preferably a C₁₆-C₂₀ alkyl group, R² is a methyl or alkyl group, and R³ and R⁴, which may be the same or different, are methyl or hydroxyethyl groups. The fatty ammonium is preferably octadecyl trimethyl ammonium.

Active herein comprises various skin benefit agents such as anti-mosquito agents to provide mosquito repellence benefit and fragrance to provide a favorable sensory for the skin cleansing composition.

Various actives can be incorporated into the particles. The actives are selected from natural essential oil, pro-fragrance, fragrance, mosquito repellence active, anti-dandruff active or a mixture thereof, preferably the actives are mosquito repellence actives. Preferably the active has a log P value from 1.5 to 20.

Mosquito repellence actives suitable for use in the composition comprise citronella oil, peppermint oil, eucalyptus oil, lavender oil, bergamot oil, lemongrass oil, N, N-diethyl-meta-toluamide (DEET), or a mixture thereof. Preferably the active is citronella oil, peppermint oil, eucalyptus oil, lavender oil, bergamot oil or a mixture thereof. More preferably the active is citronella oil.

Typical anti-dandruff actives suitable for use in the composition comprise metal pyrithione, e.g. zinc pyrithione (ZPTO), Octopirox® (piroctone olamine), azole antimicrobials (e.g. climbazole), selenium sulfide and combinations thereof. Additionally, some natural actives such as hinokitiol or willow bark extract may also be used in hair care composition to alleviate the dandruff condition.

Typical fragrance components which is advantageous to employ in the embodiments of the present invention include those which has a relatively low boiling point, preferably those with a boiling point at less than 300° C., preferably 100-250° C., measure at 1 atmosphere.

The pro-fragrance can be a lipid. Lipids typically comprise structural units with pronounced hydrophobicity. The majority of lipids are derived from fatty acids. In these ‘acyl’ lipids, the fatty acids are predominantly present as esters and include mono-, di-, triacyl glycerols, phospholipids, glycolipids, diol lipids, waxes, sterol esters and tocopherols.

The weight ratio of the particle to the active is in the range of 1:8 to 5:1, preferably 1:3 to 3:1.

### Surfactant

The composition comprises an anionic surfactant. More than one anionic surfactant may be used in the composition. The anionic surfactant may be chosen from soap surfactant, non-soap anionic and mixtures thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred.

Preferably non-soap anionic surfactant are alkyl ether sulfates, alkyl sulfates, or a mixture thereof, more preferably sodium lauryl ether sulfate and sodium lauryl sulfate or a mixture thereof, and most preferably sodium lauryl ether sulfate.

It is preferred that the level of sodium lauryl ether sulfate is from 0% to 30%, more preferably from 1% to 25%, and most preferably from 2% to 15%, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). The level of non-ionic surfactant is preferably from 0% to 30%, more preferably from 1% to 25%, and most preferably from 2% to 15%, by weight of the composition.

Suitable amphoteric surfactants are preferably betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0% to 2%, more preferably from 1% to 10%, by weight of the composition.

### Other Ingredients

The composition may further comprise conditioners and moisturizers such as water-insoluble skin benefit agents. Examples of water-insoluble skin benefit agents include silicone oils; hydrocarbons such as liquid paraffins, petrolatum, microcrystalline wax, and mineral oil; and vegetable triglycerides such as sunflower seed and cottonseed oils.

Thickeners may also be formulated in some compositions. Thickeners may be selected from cellulosics, natural gums and acrylic polymers but not limited to these agents. Cellulosics, for example, sodium carboxymethyl cellulose, hydroxyethyl cellulose, hydroxypropyl methylcellulose and combinations thereof may be used. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01% to 3% by weight of the active polymer (outside of solvent or water) in the compositions.

It is desirable that the compositions comprise preservatives to prevent the growth of potentially harmful microorganisms. Suitable traditional preservatives are alkyl esters of para-hydroxybenzoic acid. Other preservatives include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate and benzyl alcohol. The preservatives should be selected regarding the use of the composition and possible incompatabilities between the preservatives and other ingredients. Preservatives are preferably employed in amount ranging from 0.01% to 2% by weight of the composition.

The compositions may also comprise a variety of other optional materials. For example, antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; skin glowing agent such as Vitamin B3 compounds; antioxidative agents such as Vitamin C compounds; and colorants.

The compositions may additionally include 0.5% to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful to enhance the appearance or properties of the product.

Preferably the composition comprises water in an amount of at least 5% by weight of the composition, more preferably at least 25%, even more preferably 40% to 90% and still even more preferably at least 60% to 85% by weight of the composition.

### Method and Use of the Skin Cleansing Composition

A rinse-off personal care composition as used herein includes a composition for topical application to the skin, scalp or hair of mammals, especially humans. Rinse-off is sometimes also referred to as wash-off. A rinse-off composition means a composition that is applied to the desired skin, scalp or hair surface for a shorter period, for example, a few seconds or minutes and then washed off with copious amounts of water. The rinse-off personal care composition can be skin cleansing composition or hair cleansing composition, preferably skin cleansing composition. The composition of the present invention is in the form of a liquid, lotion, cream, foam, scrub, or gel form.

The term “skin” as used herein includes the skin on the face (except eye lids and lips), neck, chest, abdomen, back, arms, under arms, hands, and legs. Preferably “skin” means includes the skin on the face (except eye lids and lips) and under arms, more preferably skin means skin on the face other than lips and eyelids. The term “hair” as used herein includes hair on the head. During application of the rinse-off personal care composition, the active is released when the microcapsule is broken by action of pressure and/or friction.

The present invention also provides a rinse-off personal care composition comprising:
(a) a microcapsule comprising an intercalated layered particle having an interlayer distance of 1 to 10 nm and an active inside of the particle; and
(b) an anionic surfactant,
for use in providing enhanced foaming benefit, wherein the active is selected from natural essential oil, pro-fragrance, fragrance, mosquito repellence active, anti-dandruff active or a mixture thereof.

The present invention also provides a combination of:
(a) a microcapsule comprising an intercalated layered particle having an interlayer distance of 1 to 10 nm and an active inside of the particle; and
(b) an anionic surfactant,
in the manufacture of a medicament for providing enhanced foaming benefit, wherein the active is selected from natural essential oil, pro-fragrance, fragrance, mosquito repellence active, anti-dandruff active or a mixture thereof.

Preferably, the method is non-therapeutic. Preferably the use is non-therapeutic. The term non-therapeutic means for cosmetic purposes and not curative or therapeutic purposes.

The following examples are provided to facilitate an understanding of the invention. The examples are not intended to limit the scope of the claims.

### EXAMPLES

### Materials:

TABLE 1

| Material | Tradename | Supplier |
|---|---|---|
| Citronella oil | Citronella oil | Jian Zhongxiang natural plant Co. Ltd |
| Intercalated | HFGEL-140F | Zhejiang Fenghong new material Co. Ltd |
| bentonite | | |

### Example 1

This example demonstrates the foaming test of samples.

A series of microcapsules were prepared according to Table 1 using the procedure as follows. 1.25 g of citronella oil was added into 2.5 g of capsule material in a container and stirred vigorously for 5 mins when all liquid was absorbed by the capsule material. The container was then sealed for further use.

TABLE 1

| | Samples (wt %) | | |
|---|---|---|---|
| Ingredient | a | b | c |
| Citronella oil | 33.3 | 33.3 | 33.3 |
| Boron Nitride | 66.7 | — | — |
| Bentonite | — | 66.7 | — |
| Intercalated Bentonite | — | — | 66.7 |

A series of body wash compositions were prepared according to Table 2. For sample D, citronella oil and intercalated bentonite were directly mixed with SLES and DI water.

TABLE 2

| | Samples | | | | |
|---|---|---|---|---|---|
| Ingredient | A | B | C | 1 | D |
| Citronella oil | 2.5 | — | — | — | 2.5 |
| Sample a | — | 7.5 | — | — | — |
| Sample b | — | — | 7.5 | — | — |
| Sample c | — | — | — | 7.5 | — |
| Intercalated Bentonite | — | — | — | — | 5.0 |
| SLES | 11.0 | 11.0 | 11.0 | 11.0 | 11.0 |
| DI water | to 100 | to 100 | to 100 | to 100 | to 100 |

The foaming volume was evaluated by the procedure as follows. In a 50 mL measuring cylinder, 1 mL of sample and 9 mL of DI water were added. After sealed with a plug, the cylinder with sample slurry was shaken up and down for 20 times. After 2-min settling, the total volume was recorded. Foaming volume was calculated by the following equation:

Foaming volume=Total volume−10

TABLE 3

| | A | B | C | 1 | D |
|---|---|---|---|---|---|
| Foaming volume (mL) | 21.7 | 18.7 | 19.7 | 28.3 | 20.3 |

Table 3 shows the foaming volume of samples. It was surprisingly found that the present invention (sample 1) had higher foaming volume than comparative samples.

1. A rinse-off personal care composition comprising:
(a) a microcapsule comprising an intercalated layered particle having an interlayer distance of 1 to 10 nm and an active inside of the particle; and
(b) an anionic surfactant,
wherein the active is natural essential oil, pro-fragrance, fragrance, mosquito repellence active, anti-dandruff active or a mixture thereof; and further wherein the particle is intercalated with fatty ammonium.

2. The composition according to claim 1, wherein the particle is intercalated clay.

3. The composition according to claim 1, wherein the fatty ammonium comprises octadecyl trimethyl ammonium.

4. The composition according to claim 1, wherein the active comprises citronella oil.

5. The composition according to claim 1, wherein the weight ratio of the particle to the active is in a range from 1:8 to 5:1.

6. The composition according to claim 1, wherein the composition comprises alkyl ether sulfates, alkyl sulfates, or a mixture thereof.

7. The composition according to claim 1, wherein the interlayer distance is from 2 to 8 nm.

8. The composition according to claim 1, wherein the particle has a non-spherical shape with a sphericity of 0.01 to 0.15.

9. The composition according to claim 1, wherein the microcapsule is not a core-shell structure.

10. The composition according to claim 1, wherein the microcapsule is present in amount of 0.1% to 2% by weight of the composition.

11. A method of for foam enhancement for cleansing skin, scalp or hair of an individual comprising the steps of applying the composition according to claim 1 to the skin, scalp or hair of an individual and rising the composition off with water.

12. The method according to claim 11 wherein the composition further comprises anionic surfactant for providing foam enhancement for cleansing skin, scalp or hair of the individual.

13. The composition according to claim 1 wherein the composition further comprises alkyl betaine, alkylamido betaine, alkyl sulfobetaine, alkyl sultaine, alkylamido sultaine or a mixture thereof.

13. The composition according to claim 1 wherein the composition further comprises vitamin C, vitamin B3, menthol or a mixture thereof.
